(19) United States
(12) Patent Application Publication
(10) Pub. No.: US 2022/0252490 A1
(43) Pub. Date: Aug. 11, 2022

(54) Evaluation Method for Hydrogen Embrittlement of Rebar

(71) Applicant: Nippon Telegraph and Telephone Corporation, Tokyo (JP)

(72) Inventors: Takuya Kamisho, Musashino-shi, Tokyo (JP); Ryuta Ishii, Musashino-shi, Tokyo (JP); Yosuke Takeuchi, Musashino-shi, Tokyo (JP); Masayuki Tsuda, Musashino-shi, Tokyo (JP)

(21) Appl. No.: 17/618,369

(22) PCT Filed: Jun. 26, 2019
(86) PCT No.: PCT/JP2019/025341

§ 371 Date: Dec. 10, 2021

(51) Int. Cl. G01N 3/08; G01N 33/20

(52) U.S. Cl. CPC G01N 3/08; G01N 2203/0017; G01N 33/20

(57) ABSTRACT

Provided is a method for evaluating a hydrogen embrittlement fracture risk of an iron reinforcing bar that is performed by a hydrogen embrittlement fracture risk evaluation apparatus, the method including: a fracture probability curved surface generation step of obtaining a fracture probability curved surface representing a probability of the iron reinforcing bar fracturing by performing regression analysis on results obtained by repeatedly carrying out a hydrogen embrittlement test while changing an amount of hydrogen absorbed in the iron reinforcing bar provided in a concrete structure and a tensile stress applied to the iron reinforcing bar and using the amount of hydrogen and the tensile stress as variables; a lower limit stress acquisition step of acquiring, from the fracture probability curved surface, a lower limit stress property representing a relationship between a lower limit stress that is a lower limit of the tensile stress at which no fracture occurs in the iron reinforcing bar at a predetermined probability and the amount of hydrogen; and an evaluation step.

### TECHNICAL FIELD

The present invention relates to a method for evaluating a hydrogen embrittlement fracture risk of an iron reinforcing bar provided in a concrete structure.

### BACKGROUND ART

For example, iron reinforcing bars are provided in concrete poles such as utility poles. Deterioration of the iron reinforcing bars may degrade the strength of the concrete poles, eventually cause embrittlement fracture that is fracturing of embrittled iron reinforcing bars, and lead to the concrete poles collapsing. It is thus necessary to evaluate degrees of deterioration of the iron reinforcing bars in order to maintain healthy concrete poles.

Fracture of an iron reinforcing bar can be inspected by, for example, a method using magnetism as disclosed in Non Patent Literature 1. However, it is too late to perform inspection if fracture of the iron reinforcing bar has already occurred.

It is already known that the cause of embrittlement fracture of an iron reinforcing bar is hydrogen in the iron reinforcing bar (high-strength steel) as disclosed in Non Patent Literature 2. Also, it is also known that there is a value (lower limit stress) of a tensile stress at which no hydrogen embrittlement fracture due to a minute amount of hydrogen in steel occurs. It is thus possible to evaluate the risk of hydrogen embrittlement fracture occurring if a stress applied to a concrete pole in an actual environment can be compared with a lower limit stress thereof.

### CITATION LIST

### Non Patent Literature

Non Patent Literature 1: Rebar Diagnostic CP Checker M (accessed on Jun. 10, 2019), Internet (URL: http://www.ssken.co.jp/service/cpc.html)
Non Patent Literature 2: Shinichi Suzuki and three others, “Estimation of Delayed Fracture Property of Steels”, Iron and Steel, vol. 79, No. 2, 1992

### SUMMARY OF THE INVENTION

### Technical Problem

However, in a case in which a lower limit stress in an actual environment is evaluated, it takes a long time such as several decades until fracture occurs if a test is carried out with an amount of hydrogen that is as small as that in an actual environment, and it is thus not possible to actually perform the evaluation. Also, since hydrogen embrittlement fracture occurs randomly, a stress at which fracture does not occur also varies randomly, and it is thus not possible to evaluate the lower limit stress with high accuracy. In other words, there is a problem that there are no evaluation methods that enable appropriate evaluation of a risk of hydrogen embrittlement fracture of an iron reinforcing bar in the related art.

The present invention was made in view of these problems, and an object thereof is to provide a method for evaluating a hydrogen embrittlement fracture risk of an iron reinforcing bar that enables appropriate evaluation of a risk of hydrogen embrittlement fracture of the iron reinforcing bar.

### Means for Solving the Problem

According to an aspect of the present invention, there is provided a method for evaluating a hydrogen embrittlement fracture risk of an iron reinforcing bar that is performed by a hydrogen embrittlement fracture risk evaluation apparatus, the method including: obtaining a fracture probability curved surface representing a probability of the iron reinforcing bar fracturing by performing regression analysis on results obtained by repeatedly carrying out a hydrogen embrittlement test while changing an amount of hydrogen absorbed in the iron reinforcing bar provided in a concrete structure and a tensile stress applied to the iron reinforcing bar and using the amount of hydrogen and the tensile stress as variables; acquiring, from the fracture probability curved surface, a lower limit stress property representing a relationship between a lower limit stress that is a lower limit of the tensile stress at which fracture does not occur in the iron reinforcing bar at a predetermined probability and the amount of hydrogen; and evaluating a risk of hydrogen embrittlement fracture of the iron reinforcing bar on the basis of the lower limit stress property and a maximum value of the tensile stress obtained from an amount of deflection of the concrete structure with the iron reinforcing bar provided therein.

### Effects of the Invention

According to the present invention, it is possible to provide an evaluation method that enables appropriate evaluation of a risk of hydrogen embrittlement fracture of an iron reinforcing bar.

### BRIEF DESCRIPTION OF DRAWINGS

FIG. 1 is a diagram illustrating a functional configuration example of a hydrogen embrittlement fracture risk evaluation apparatus according to a first embodiment of the present invention.

FIG. 2 is a diagram illustrating an example of a relationship between a concentration of ammonium thiocyanate and an equilibrium amount of hydrogen in an iron reinforcing bar.

FIG. 3 is a diagram schematically illustrating an example of a fracture probability curve representing a relationship between tensile stress and fracture.

FIG. 4 is a diagram schematically illustrating an example of a fracture probability curved surface representing a probability of the iron reinforcing bar fracturing using the amount of hydrogen and tensile stress as variables.

FIG. 5 is a diagram illustrating an example of a relationship between the amount of hydrogen and tensile stress.

FIG. 6 is a diagram illustrating a functional configuration example of a hydrogen embrittlement fracture risk evaluation apparatus according to a second embodiment of the present invention.

FIG. 7 is a diagram schematically illustrating a plurality of lower limit stress properties.

FIG. 8 is a diagram schematically illustrating a property obtained by converting a plurality of lower limit stress properties into a relationship between tensile stress and fracture probability.

FIG. 9 is a flowchart illustrating a processing procedure of a method for evaluating a hydrogen embrittlement fracture risk of an iron reinforcing bar according to the present invention.

FIG. 10 is a block diagram illustrating a configuration example of a general-purpose computer system.

### DESCRIPTION OF EMBODIMENTS

Hereinafter, embodiments of the present invention will be described with reference to the drawings. The same components in a plurality of drawings are denoted using the same reference signs, and description thereof will not be repeated.

### First Embodiment

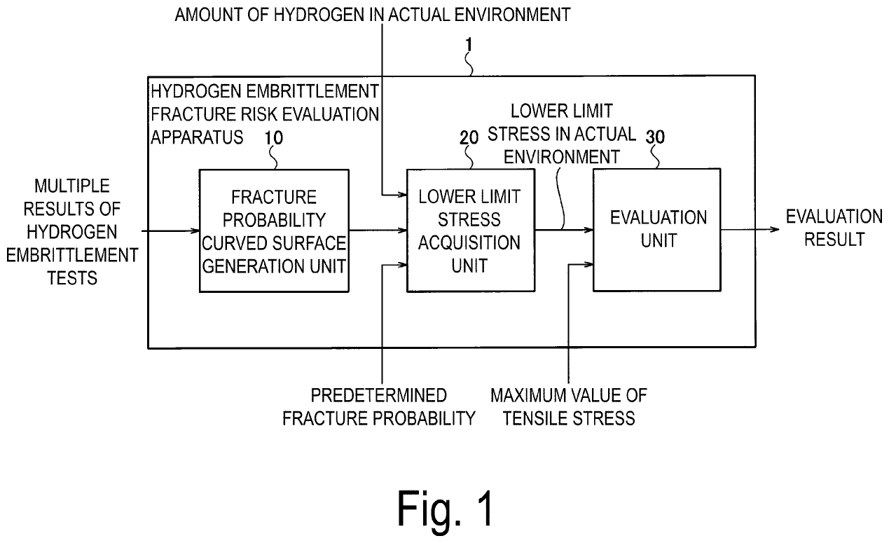

FIG. 1 is a diagram illustrating a functional configuration example of a hydrogen embrittlement fracture risk evaluation apparatus according to a first embodiment of the present invention. A hydrogen embrittlement fracture risk evaluation apparatus 1 illustrated in FIG. 1 is an apparatus configured to evaluate a risk of hydrogen embrittlement fracture of an iron reinforcing bar provided in a concrete structure.

The hydrogen embrittlement fracture risk evaluation apparatus 1 includes a fracture probability curved surface generation unit 10, a lower limit stress acquisition unit 20, and an evaluation unit 30. Each functional configuration unit of the hydrogen embrittlement fracture risk evaluation apparatus 1 can be realized by a computer including, for example, a ROM, a RAM, a CPU, and the like.

The fracture probability curved surface generation unit 10 performs regression analysis on results obtained by repeatedly carrying out a hydrogen embrittlement test while changing the amount of hydrogen absorbed by the iron reinforcing bar provided in the concrete structure and a tensile stress applied to the iron reinforcing bar and using the amount of hydrogen and the tensile stress as variables. The fracture probability aspect generation surface 10 then generates a fracture probability curved surface representing a probability of the iron reinforcing bar fracturing. Here, the amount of hydrogen absorbed by the iron reinforcing bar is adjusted by immersion of the iron reinforcing bar in each of a plurality of solutions formulated such that a substance amount corresponding to the concentration of ammonium thiocyanate in a 1 mol/L aqueous solution of sodium hydroxide is a predetermined value and causing a predetermined current to flow therethrough.

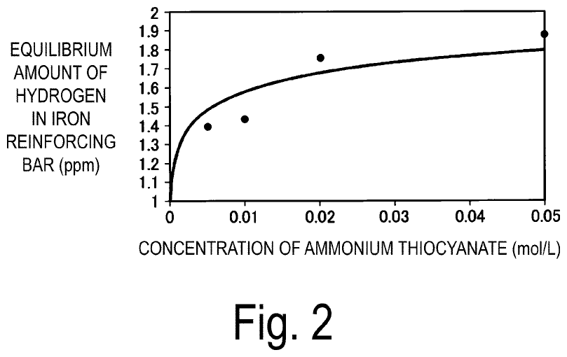

FIG. 2 is a diagram illustrating a relationship example of the concentration of ammonium thiocyanate in the 1 mol/L aqueous solution of sodium hydroxide and the equilibrium amount of hydrogen in the iron reinforcing bar. The horizontal axis in FIG. 2 represents the concentration of ammonium thiocyanate (mol/L), and the vertical axis represents the equilibrium amount of hydrogen (ppm) in the iron reinforcing bar. FIG. 2 illustrates properties in a case in which the current density is 0.01 mA/mm².

As illustrated in FIG. 2, when the substance amount corresponding to the concentration of ammonium thiocyanate in the 1 mol/L of aqueous solution of sodium hydroxide is 0.01 mol/L, the equilibrium amount of hydrogen in the iron reinforcing bar is about 1.45 ppm. Similarly, when the substance amount corresponding to the concentration of ammonium thiocyanate is 0.05 mol/L, the equilibrium amount of hydrogen in the iron reinforcing bar is about 1.9 ppm.

In the hydrogen embrittlement test, the iron reinforcing bar is dipped in the aforementioned solution, a current with a current density of 0.01 mA/mm² is caused to flow therethrough to cause hydrogen to invade the iron reinforcing bar, and a predetermined tensile stress is then applied to cause fracture. The hydrogen embrittlement test is repeatedly carried out while changing the equilibrium amount of hydrogen.

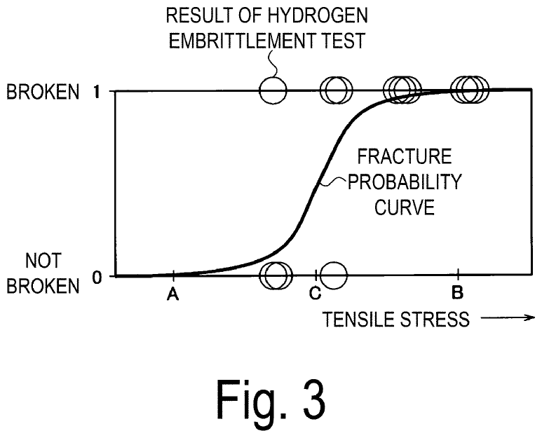

FIG. 3 is a diagram schematically illustrating a fracture probability curve generated by performing regression analysis on results obtained by repeatedly carrying out the hydrogen embrittlement test while changing the amount of hydrogen absorbed by the iron reinforcing bar and using the tensile stress as a variable. ∘ illustrated in FIG. 3 indicates results of the hydrogen embrittlement test carried out while changing the amount of hydrogen.

The fracture probability curve representing a probability of the iron reinforcing bar fracturing due to a tensile stress is obtained through regression analysis of a relationship between the tensile stress and whether or not fracture occurs. As illustrated in FIG. 3, it is possible to ascertain from the fracture probability curve that no fracture occurs with a tensile stress of equal or less than A, fracture occurs with a tensile stress of equal to or greater than B, and the fracture probability with a tensile stress C is, for example, 50%. The fracture probability can be represented by the following equation, for example.

$$\begin{matrix}Math . 1 & \\ FRACTURE PROBABILITY = \frac{1}{1 + exp ( - {β}_{0} - {β}_{1} \times TENSILE STRESS} & ( 1 )\end{matrix}$$

B₀ and B₁ are coefficients of a logistic function. Note that the fracture probability may not cause regression in the logistic function. For example, the fracture probability may cause regression in a sigmoid function or a probit function.

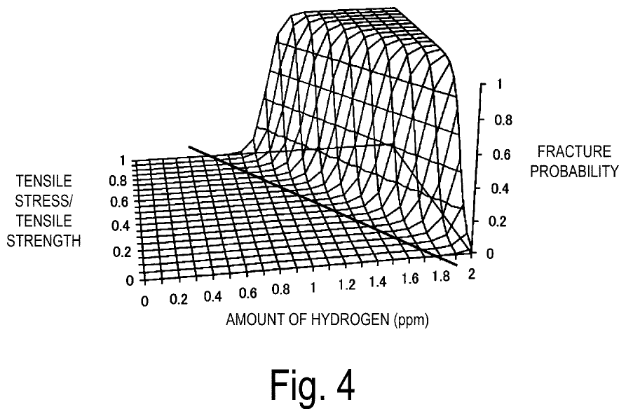

If the amount of hydrogen is added to variables in the fracture probability curve, and regression analysis is performed on the results obtained by repeatedly carrying out the hydrogen embrittlement test while changing the amount of hydrogen and using the amount of hydrogen and the tensile strength as variables, then a fracture probability curved surface can be generated. FIG. 4 is a diagram schematically illustrating an example of the fracture probability curved surface representing the probability of the iron reinforcing bar fracturing, using the amount of hydrogen and the tensile stress as variables. As illustrated in FIG. 4, the x axis in the x-y plane represents the amount of hydrogen (ppm), the y axis represents the tensile stress, and the z axis represents the fracture probability. The fracture probability in a case in which hydrogen is added to the variables can be represented by the following equation.

$$\begin{matrix}Math . 2 & \\ FRACTURE PROBABILITY = \frac{1}{1 + exp ( - {β}_{0} - {β}_{1} \times TENSILE STRESS - {β}_{2} \times AMOUNT OF HYDROGEN} & ( 2 )\end{matrix}$$

The lower limit stress acquisition unit 20 acquires a lower limit stress that is a value of a tensile stress with which no fracture occurs in the iron reinforcing bar in association with the amount of hydrogen. In FIG. 4, the lower limit stress at the fracture probability of 0.01% corresponding to the amount of hydrogen is a tensile stress represented by the thick solid line connecting intersecting points of the x-y plane at the z axis of 0.0001. The following description will be given under the assumption that the allowable fracture probability (lower limit stress) is 0.01%.

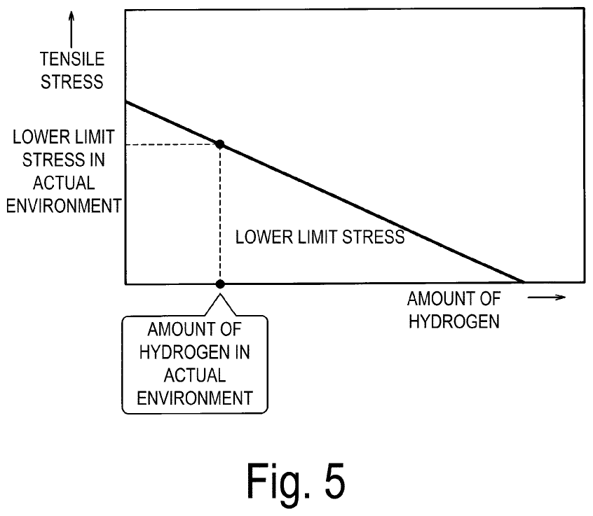

FIG. 5 is a diagram schematically illustrating a change in lower limit stress with respect to the thus obtained amount of hydrogen. In FIG. 5, the value of the tensile stress with respect to the amount of hydrogen absorbed by the iron reinforcing bar in the actual environment is the lower limit stress (the value of the tensile stress with which no fracture occurs in the iron reinforcing bar) in the actual environment.

The lower limit stress acquisition unit 20 acquires the lower limit stress for a predetermined fracture probability with respect to the amount of hydrogen absorbed in the iron reinforcing bar in the actual environment. The property illustrated in FIG. 5 is, for example, a lower limit stress for the fracture probability of 0.01%.

The amount of hydrogen absorbed in the iron reinforcing bar in the actual environment is obtained from an actual concrete structure. The amount of hydrogen in the actual environment can be obtained by analyzing, for example, an iron reinforcing bar of an aged concrete structure by using a thermal desorption analysis apparatus.

The evaluation unit 30 compares a lower limit stress acquired by the lower limit stress acquisition unit 20 with a maximum value of a tensile stress obtained from an amount of deflection of the concrete structure with the iron reinforcing bar provided therein. The maximum value of the tensile stress is obtained by applying a load to the concrete structure to deflect the concrete structure, measuring the distortion of the iron reinforcing bar, and multiplying the distortion by the elastic modulus of the iron reinforcing bar. Also, the maximum value of the tensile stress may be obtained through numerical calculation using a definite element method.

It is possible to evaluate a risk of hydrogen embrittlement fracture being caused in the iron reinforcing bar by, for example, comparing the maximum value of the tensile stress with the lower limit stress for the fracture probability of 0.01%. If the maximum value of the tensile stress applied to the concrete structure is smaller than the lower limit stress for the fracture probability of 0.01%, it is possible to determine that there is no risk of hydrogen embrittlement fracture. Also, if the maximum value of the tensile stress is greater than the lower limit stress for the fracture probability of 0.01%, it is possible to determine that there is a risk of hydrogen embrittlement fracture.

As described above, the hydrogen embrittlement fracture risk evaluation apparatus 1 according to the present embodiment includes the fracture probability curved surface generation unit 10, the lower limit stress acquisition unit 20, and the evaluation unit 30. The fracture probability curved surface generation unit 10 generates a fracture probability curved surface representing the probability of the iron reinforcing bar fracturing by performing regression analysis on results obtained by repeatedly carrying out the hydrogen embrittlement test while changing the amount of hydrogen absorbed by the iron reinforcing bar provided in the concrete structure and the tensile stress applied to the iron reinforcing bar and using the amount of hydrogen and the tensile stress as variables. The lower limit stress acquisition unit 20 acquires, from the fracture probability curved surface, a lower limit stress property representing a relationship between the amount of hydrogen and the lower limit stress that is a lower limit of the tensile stress at which no fracture occurs in the iron reinforcing bar at a predetermined probability, and then obtains a lower limit stress with respect to the amount of hydrogen in the actual environment from the lower limit stress property. The evaluation unit 30 compares the lower limit stress obtained by the lower limit stress acquisition unit 20 with the maximum value of the tensile stress obtained from the amount of deflection of the concrete structure with the iron reinforcing bar provided therein, and evaluates that there is no risk of hydrogen embrittlement fracture of the iron reinforcing bar if the lower limit stress is smaller than the maximum value of the tensile stress, or evaluates that there is a risk of hydrogen embrittlement fracture of the iron reinforcing bar if the lower limit stress is greater than the maximum value of the tensile stress. It is thus possible to evaluate the risk of hydrogen embrittlement fracture of the iron reinforcing bar with two values, namely, whether or not fracture will occur.

### Second Embodiment

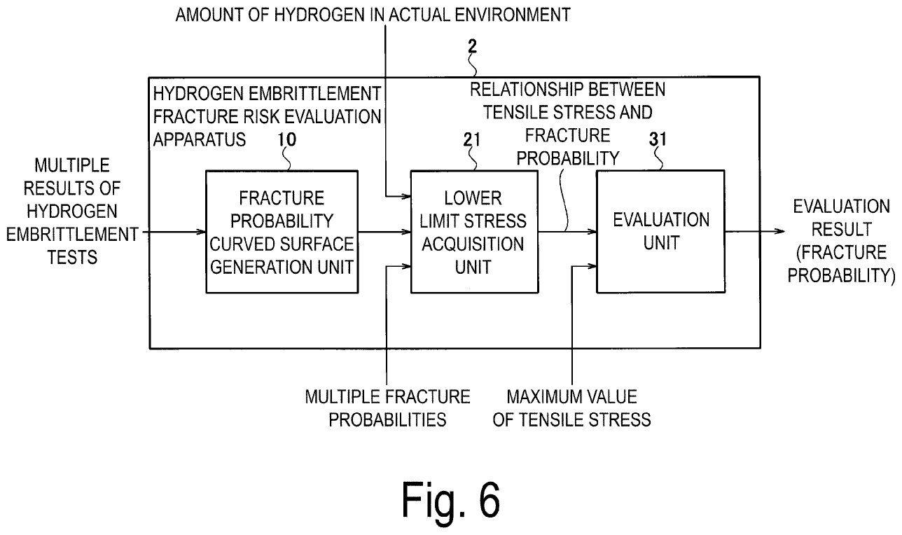

FIG. 6 is a diagram illustrating a functional configuration example of a hydrogen embrittlement fracture risk evaluation apparatus according to a second embodiment of the present invention. A hydrogen embrittlement fracture risk evaluation apparatus 2 illustrated in FIG. 1 is different from the hydrogen embrittlement fracture risk evaluation apparatus 1 (FIG. 1) in that the hydrogen embrittlement fracture risk evaluation apparatus 2 includes a lower limit stress acquisition unit 21 and an evaluation unit 31. The lower limit stress acquisition unit 21 and the evaluation unit 31 are functional configuration units corresponding to the lower limit stress acquisition unit 20 and the evaluation unit 30 in the hydrogen embrittlement fracture risk evaluation apparatus 1, respectively.

The lower limit stress acquisition unit 21 acquires lower limit stress properties for a plurality of fracture probabilities and compares the acquired plurality of lower limit stress properties with a relationship between fracture probability and tensile stress with respect to the amount of hydrogen absorbed in the iron reinforcing bar in the actual environment. The plurality of fracture probabilities are, for example, 0.01%, 20%, 40%, 60%, 80%, and 99.99%.

The lower limit stress acquisition unit 21 acquires the lower limit stress properties for the fracture probabilities of 0.01%, 20%, 40%, 60%, 80%, and 99.99%, for example, in comparison with the lower limit stress acquisition unit 20 (FIG. 1) configured to acquire only the lower limit stress property of the fracture probability of 0.01%.

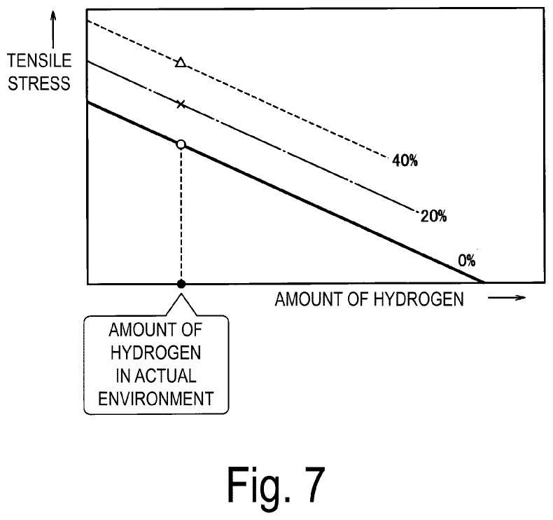

FIG. 7 is a diagram schematically illustrating a plurality of lower limit stress properties. The relationship between the horizontal axis and the vertical axis is the same as that in FIG. 5. The solid line represents the lower limit stress property for the fracture probability of 0.01%. The one-dot dashed line represents the lower limit stress property for the fracture probability of 20%. The broken line represents the lower limit stress property for the fracture probability of 40%. Illustration of the lower limit stress properties for the fracture probabilities of 60%, 80%, and 99.99% has been omitted.

The lower limit stress property for the fracture probability of 20% is a property represented by plotting the x-y coordinates with the z axis of 0.2 in FIG. 4. The lower limit stress property for the fracture probability of 40% is a property represented by plotting the x-y coordinates with the z axis of 0.4 in FIG. 4.

The lower limit stress acquisition unit 21 converts the plurality of lower limit stress properties acquired as described above into a relationship between the fracture probability and tensile stress with respect to the amount of hydrogen absorbed by the iron reinforcing bar in the actual environment. The conversion is performed by obtaining the tensile stress of each lower limit stress property with respect to the amount of hydrogen absorbed by the iron reinforcing bar in the actual environment for each fracture probability.

As illustrated in FIG. 7, the tensile stress ∘, ×, or Δ for each fracture probability with respect to the amount of hydrogen absorbed by the iron reinforcing bar in the actual environment is plotted on two-dimensional coordinates of the tensile stress (x) and the fracture probability (y). The plotted properties represent the fracture probabilities with respect to the tensile stress.

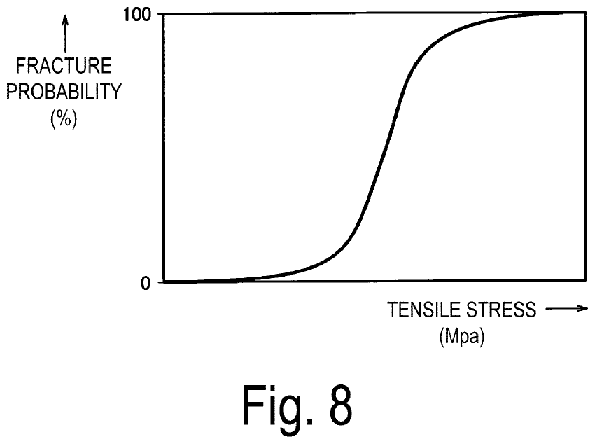

FIG. 8 illustrates a property obtained by converting the plurality of lower limit stress properties into a relationship between tensile stress and fracture probability. The horizontal axis represents the tensile stress (MPa), and the vertical axis represents the fracture probability (%).

The evaluation unit 31 obtains the fracture probability with respect to the maximum value of the tensile stress from the relationship between tensile stress and fracture probability converted by the lower limit stress acquisition unit 21. Here, the maximum value of the tensile stress is a maximum value of the tensile stress applied to the actual concrete structure. The evaluation unit 31 can thus obtain the fracture probability with respect to the maximum value of the tensile stress.

As described above, the hydrogen embrittlement fracture risk evaluation apparatus 2 according to the present embodiment includes the lower limit stress acquisition unit 21 configured to acquire the lower limit stress properties of the plurality of fracture probabilities and convert the plurality of lower limit stress properties into a relationship between tensile stress and fracture probability with respect to the amount of hydrogen in the actual environment and the evaluation unit 31 configured to obtain the fracture probability with respect to the maximum value of the tensile stress from the converted relationship between the tensile stress and the fracture probability. It is thus possible to evaluate a risk of hydrogen embrittlement fracture of the iron reinforcing bar with the fracture probability.

### Method for Evaluating Hydrogen Embrittlement Fracture Risk of Iron Reinforcing Bar

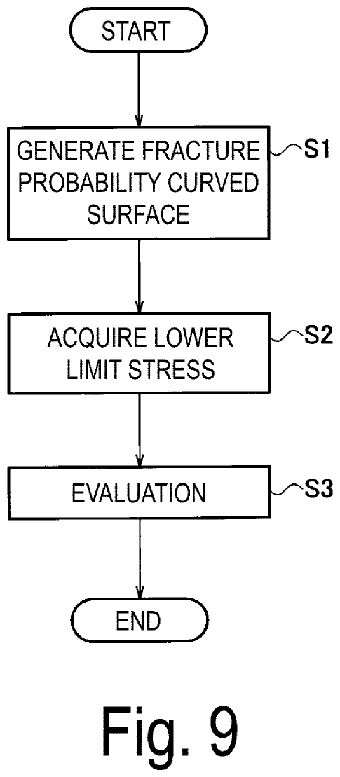

The aforementioned hydrogen embrittlement fracture risk evaluation apparatuses 1 and 2 execute the method for evaluating a hydrogen embrittlement fracture risk of the processing procedure illustrating in FIG. 9. The method for evaluating a hydrogen embrittlement fracture risk of an iron reinforcing bar will be described with reference to FIG. 9.

The fracture probability curved surface generation unit 10 generates a fracture probability curved surface representing a probability of an iron reinforcing bar fracturing (Step S1) by performing regression analysis on results obtained by repeatedly carrying out a hydrogen embrittlement test while changing the amount of hydrogen absorbed by an iron reinforcing bar provided in a concrete structure and a tensile stress applied to the iron reinforcing bar and using the amount of hydrogen and the tensile stress as variables.

The lower limit stress acquisition unit 20 acquires, from the fracture probability curved surface, a lower limit stress property representing a relationship between the amount of hydrogen and the lower limit stress that is a lower limit of the tensile stress at which no fracture occurs in the iron reinforcing bar at a predetermined probability (Step S2).

The evaluation unit 30 evaluates the risk of hydrogen embrittlement fracture of the iron reinforcing bar based on the lower limit stress property and the maximum value of the tensile stress obtained from the amount of deflection of the concrete structure with the iron reinforcing bar provided therein (Step S3).

According to the method for evaluating a hydrogen embrittlement fracture risk of an iron reinforcing bar of the present embodiment, it is possible to appropriately evaluate a risk of hydrogen embrittlement fracture of the iron reinforcing bar.

Also, the lower limit stress acquisition step according to the first embodiment of the present invention includes obtaining the lower limit stress with respect to the amount of hydrogen in the actual environment from the lower limit stress property, and the evaluation step includes comparing the lower limit stress obtained in the lower limit stress acquisition step with the maximum value of the tensile stress obtained from the amount of deflection of the concrete structure with the iron reinforcing bar provided therein, and evaluating that there is no risk of hydrogen embrittlement fracture of the iron reinforcing bar if the lower limit stress is smaller than the maximum value of the tensile stress, or evaluating that there is a risk of hydrogen embrittlement fracture of the iron reinforcing bar if the lower limit stress is greater than the maximum value of the tensile stress. It is thus possible to evaluate the risk of hydrogen embrittlement fracture of the iron reinforcing bar with two values, namely, whether or not fracture will occur.

Also, the lower limit stress acquisition step according to the second embodiment of the present invention includes acquiring lower limit stress properties of the plurality of fracture probabilities and converting the plurality of lower limit stress properties into a relationship between fracture probability and tensile stress with respect to the amount of hydrogen in the actual environment, and the evaluation step includes obtaining the fracture probability with respect to the maximum value of the tensile stress from the converted relationship between tensile stress and the fracture probability. It is thus possible to evaluate a risk of hydrogen embrittlement fracture of the iron reinforcing bar with the fracture probability.

According to the hydrogen embrittlement fracture risk evaluation apparatuses 1 and 2, it is possible to evaluate a risk of hydrogen embrittlement fracture of an iron reinforcing bar provided in a concrete structure (for example, a utility pole) with a lower limit stress as described above.

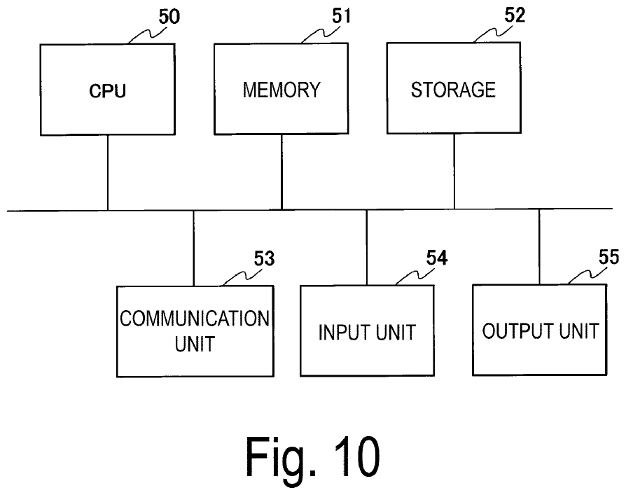

The hydrogen embrittlement fracture risk evaluation apparatuses 1 and 2 can be realized by a general-purpose computer system illustrated in FIG. 10. For example, each function of the hydrogen embrittlement fracture risk evaluation apparatuses 1 and 2 are realized by a CPU 50 executing a predetermined program loaded on a memory 51 in a general-purpose computer system including the CPU 50, the memory 51, a storage 52, a communication unit 53, an input unit 54, and an output unit 55. The predetermined program can also be recorded in a computer readable recording medium such as an HDD, an SSD, a USB memory, a CD-ROM, a DVD-ROM, or an MO or can be distributed via a network.

The present invention is not limited to the aforementioned embodiments, and modifications can be made within the gist thereof. Although the example in which regression is achieved with the logistic function in the regression analysis has been described above, for example, regression may be achieved with another function.

It is a matter of course that various embodiments and the like that are not described herein are also included in the present disclosure. Thus, the technical scope of the present invention is defined only by the subject matters according to the claims that are appropriate from the description above.

### REFERENCE SIGNS LIST

1, 2 Hydrogen embrittlement fracture risk evaluation apparatus
10 Fracture probability curved surface generation unit
20, 21 Lower limit stress acquisition unit
30, 31 Evaluation unit

1. A method for evaluating a hydrogen embrittlement fracture risk of an iron reinforcing bar that is performed by a hydrogen embrittlement fracture risk evaluation apparatus, the method comprising:
obtaining a fracture probability curved surface representing a probability of the iron reinforcing bar fracturing by performing regression analysis on results obtained by repeatedly carrying out a hydrogen embrittlement test while changing an amount of hydrogen absorbed in the iron reinforcing bar provided in a concrete structure and a tensile stress applied to the iron reinforcing bar and using the amount of hydrogen and the tensile stress as variables;
acquiring, from the fracture probability curved surface, a lower limit stress property representing a relationship between a lower limit stress that is a lower limit of the tensile stress at which no fracture occurs in the iron reinforcing bar at a predetermined probability and the amount of hydrogen; and
evaluating a risk of hydrogen embrittlement fracture of the iron reinforcing bar on the basis of the lower limit stress property and a maximum value of the tensile stress obtained from an amount of deflection of the concrete structure with the iron reinforcing bar provided therein.

2. The method for evaluating a hydrogen embrittlement fracture risk of an iron reinforcing bar according to claim 1,
wherein in the acquiring of the lower limit stress, a lower limit stress with respect to the amount of hydrogen in an actual environment is obtained from the lower limit stress property, and
in the evaluating of the risk, the lower limit stress obtained in the acquiring of the lower limit stress is compared with the maximum value of the tensile stress obtained from the amount of deflection of the concrete structure with the iron reinforcing bar provided therein, and it is evaluated that there is no risk of hydrogen embrittlement fracture of the iron reinforcing bar if the lower limit stress is smaller than the maximum value of the tensile stress, and it is evaluated that there is a risk of hydrogen embrittlement fracture of the iron reinforcing bar if the lower limit stress is greater than the maximum value of the tensile stress.

3. The method for evaluating a hydrogen embrittlement fracture risk of an iron reinforcing bar according to claim 1,
wherein in the acquiring of the lower limit stress, a plurality of the lower limit stress properties for a plurality of fracture probabilities are acquired, and the plurality of the lower limit stress properties are converted into a relationship between tensile stress and fracture probability with respect to the amount of hydrogen in an actual environment, and
in the evaluating of the risk, a fracture probability with respect to the maximum value of the tensile stress is obtained from the converted relationship between tensile stress and fracture probability.

4. The method for evaluating a hydrogen embrittlement fracture risk of an iron reinforcing bar according to claim 1, wherein a plurality of the amounts of hydrogen used in the hydrogen embrittlement test are adjusted by immersion in a plurality of solutions formulated such that a substance amount corresponding to a concentration of ammonium thiocyanate in a 1 mol/L aqueous solution of sodium hydroxide is a predetermined value.

5. The method for evaluating a hydrogen embrittlement fracture risk of an iron reinforcing bar according to claim 2, wherein a plurality of the amounts of hydrogen used in the hydrogen embrittlement test are adjusted by immersion in a plurality of solutions formulated such that a substance amount corresponding to a concentration of ammonium thiocyanate in a 1 mol/L aqueous solution of sodium hydroxide is a predetermined value.

6. The method for evaluating a hydrogen embrittlement fracture risk of an iron reinforcing bar according to claim 3, wherein a plurality of the amounts of hydrogen used in the hydrogen embrittlement test are adjusted by immersion in a plurality of solutions formulated such that a substance amount corresponding to a concentration of ammonium thiocyanate in a 1 mol/L aqueous solution of sodium hydroxide is a predetermined value.
